(19) United States
(12) Patent Application Publication
(10) Pub. No.: US 2019/0233153 A1
(43) Pub. Date: Aug. 1, 2019

(54) MANUFACTURING AND PACKAGING OF A STERILE DRUG PRODUCT

(71) Applicant: AL Pharma, Inc., Oklahoma City, OK (US)

(72) Inventor: John Hofstetter, Nobel, OK (US)

(21) Appl. No.: 16/256,759

(22) Filed: Jan. 24, 2019

Related U.S. Application Data
(60) Provisional application No. 62/622,269, filed on Jan. 26, 2018.

(51) Int. Cl. B65B 55/12; B65B 55/04; A61J 1/00

(52) U.S. Cl. CPC B65B 55/12; B65B 55/04; B65B 3/003; A61L 2202/18; A61J 1/00

(57) ABSTRACT

The present invention is directed to a process of manufacturing a pharmaceutical composition, a pharmaceutical composition produced by the process of the present invention and a container comprising a pharmaceutical composition produced by the process of the present invention.

### FIELD OF THE INVENTION

The present invention is related to a process of manufacturing a pharmaceutical composition, a pharmaceutical composition produced by the process of the present invention and a container comprising a pharmaceutical composition produced by the process of the present invention.

### BACKGROUND OF THE INVENTION

It is a well-accepted principle that sterile drugs should be manufactured using aseptic processing only when terminal sterilization is not feasible. The reason for this is approach is that lack of sterility assurance is the primary reason for drug recalls. Nearly all drugs recalled due to lack of sterility assurance in the last twenty years were produced via aseptic processing (i.e. sterile filtration). By contrast the use of terminal sterilization has historically resulted in substantially lower sterility failures. While there are no absolute Food and Drug Administration standards for sterilization processes, pharmaceutical solutions are most commonly sterilized using a heating regimen at 121.1° C. for about 30 minutes. While this may be an effective method for thermally stable compounds, this practice is counterproductive for some heat-sensitive active pharmaceutical ingredients (“APIs”). In these cases, the resulting solution may be sterile, but it is often plagued with an unacceptable increase in degradation products brought on by the excessive use of heat in the sterilization process. Furthermore, compositions containing heat-sensitive APIs are often not terminally sterilized to avoid this degradation. Therefore, it is desirable to find and implement a sterilization method that utilizes less harsh conditions in order to prevent this thermal degradation from taking place, while continuing to meet sterility standards.

Thus, there is a need in the art for a process of manufacturing that overcomes this heat sensitivity barrier thus allowing heat sensitive APIs to be terminally sterilized, thereby reducing the risk of sterility failures of the product.

### SUMMARY OF THE INVENTION

The present invention is directed to a process of manufacturing a pharmaceutical composition, the process comprising the following steps:
(i) adding a pharmaceutically acceptable vehicle to a batch container comprising:
   (a) a top side comprising an opening; and
   (b) a pressurizing means,
(ii) purging the batch container with nitrogen gas;
(iii) mixing the drug vehicle;
(iv) adding an active pharmaceutical ingredient (“API”) to the pharmaceutically acceptable vehicle to create the pharmaceutical composition;
(v) mixing the pharmaceutical composition until the API is dissolved in the pharmaceutically acceptable vehicle;
(vi) sealing the opening of the top side of the batch container; and
(vii) pressurizing the batch container with nitrogen gas.

The present invention is further directed to a sterile pharmaceutical composition prepared by a process of the present invention.

The present invention is further directed to a container comprising a sterile pharmaceutical composition prepared by a process of the present invention.

### DETAILED DESCRIPTION OF THE INVENTION

L-Cysteine Hydrochloride Injection, as a heat sensitive product has historically not been amenable to terminal sterilization. L-Cysteine Hydrochloride Injection, USP is intended for use only after dilution as an additive to Crystalline Amino Acid Injections to meet the intravenous amino acid nutritional requirements of infants receiving total parenteral nutrition. In premixed solutions of crystalline amino acids, cysteine is relatively unstable over time, eventually converting to insoluble cystine. To avoid such precipitation, L-Cysteine Hydrochloride Injection USP is intended to be used as an additive with crystalline Amino Acid Injections immediately prior to administration to the patient. L-Cysteine Hydrochloride Injection, USP is intended for use only after dilution as an additive to Crystalline Amino Acid Injections to meet the intravenous amino acid nutritional requirements of infants receiving total parenteral nutrition.

The present invention is directed to a process of manufacturing a pharmaceutical composition, the process comprising the following steps:
(i) adding a pharmaceutically acceptable vehicle to a batch container comprising:
   (a) a top side comprising an opening; and
   (b) a pressurizing means,
(ii) purging the batch container with nitrogen gas;
(iii) mixing the drug vehicle; determining the dissolved oxygen content of the pharmaceutically acceptable vehicle;
(iv) adding an active pharmaceutical ingredient (“API”) to the pharmaceutically acceptable vehicle to create the pharmaceutical composition;
(v) mixing the pharmaceutical composition until the API is dissolved in the pharmaceutically acceptable vehicle;
optionally, removing a portion of the pharmaceutical composition through a pharmaceutical composition removal means on the bottom side of the batch container;
optionally, adding the removed pharmaceutical composition to the opening of the top side of the batch container;
optionally, adding additional pharmaceutically acceptable vehicle to the pharmaceutical composition to obtain a desired API concentration;
removing an amount of pharmaceutical composition necessary for assay and pH testing;
(ix) sealing the opening of the top side of the batch container;
(x) pressurizing the batch container with nitrogen gas; optionally, sterilizing stoppers in sterilization bags at 121° C. for 30 minutes;
optionally, wrapping filling equipment and sterilizing filling equipment at 121° C. for 30 minutes; and
optionally, washing vials with water for injection and depyrogenating vials in a depyrogenation oven.
(xi) optionally, filling a product container with the pharmaceutical composition from the batch container using filling equipment;
(xii) optionally, stoppering the product container; and
(xiii) optionally, capping the product container.

In a preferred embodiment, the API is L-cysteine hydrochloride monohydrate, preferably at a concentration of about 50 milligrams per milliliter.

In another preferred embodiment, the wherein the pharmaceutically acceptable vehicle is water for injection.

The present invention is further directed to a sterile pharmaceutical composition prepared by a process of the present invention.

In a preferred embodiment, sterile pharmaceutical compositions of the present invention have a pH from about 1.0 to about 2.5.

In another preferred embodiment, sterile pharmaceutical compositions of the present invention comprise less than about 1.2% cystine.

The present invention is further directed to a container comprising a sterile pharmaceutical composition prepared by a process of the present invention.

As used herein the term “pharmaceutically acceptable” refers to ingredients that are not biologically or otherwise undesirable for administration to a living subject.

Pressurizing means include, but are not limited to, a port, valve or other opening capable of selectively inputting and outputting air pressure.

Removing means include, but are not limited to, a port, valve or other opening capable of selectively inputting and outputting a liquid composition.

Stoppers suitable for use in the present invention include, but are not limited to, rubber stoppers, plastic stoppers and stoppers made from a combination of metal, rubber and/or plastic.

Filling equipment is generally known in the industry.

Depyrogenation is a technique generally known in the industry.

The following examples are intended to illustrate the process of the present invention and to teach one of ordinary skill in the art how to use the process of the invention. They are not intended to be limiting in any way.

### EXAMPLES

### Example 1

### A Process for Manufacturing Sterile L-Cysteine Hydrochloride Monohydrate

Lines and equipment were checked and cleared prior to mixing a tank batch. The batch container was then labeled as “quarantine.” The batch container was tared on a scale and water for injection (“WFI”) was added to the appropriate amount. The batch container was then purged with nitrogen gas. A mixer was then inserted into the batch container and the WFI was mixed at an appropriate speed. Oxygen content of the WFI was then determined. L-cysteine hydrochloride monohydrate was added to the WFI at an appropriate concentration to create a pharmaceutical composition. 3 liters of the pharmaceutical composition was then removed from the bottom of the batch container and added to the top of the batch container. WFI was then added to an appropriate weight to create a 50 mg/mL L-cysteine hydrochloride monohydrate. Following diluting the pharmaceutical composition to the appropriate concentration, 100 mL samples were taken for assay and pH testing, of which data is shown in Example 2 below. The batch container is then sealed and pressurized with nitrogen gas to a P.S.I. of about 5.

The pharmaceutical composition manufactured by the process above was then filled into vials. Prior to filling, stoppers were sterilized in ready to sterilize bags in an autoclave at 121° for 30 minutes. The sterilized bags are then transferred to the fill room and then to the fill line. Additionally, prior to filling, equipment used for filling the vials was wrapped and sterilized in an autoclave at 121° for 30 minutes and transferred to the filling room. Finally, prior to filling, vials washed both interiorly and exteriorly using WFI in machine washing equipment and then depyrogenated using the dry heat method in a depyrogenation oven. Filling equipment and lines were then cleared and checked. Vials were then filled and stoppered within class 100 (iso 5) conditions using the appropriate equipment. Fill volume levels were checked at appropriate intervals. Vials were then transferred to a capping area via a conveyer belt. Capped vials were then transferred to a traying area via a conveyor belt. Vials were then loaded onto SS trays. Qualified vial inspectors then inspect vials for defects. Rejected products were then placed into labeled reject bins and passing product is transported to quarantine or directly to applicable vial labeling area.

### Example 2

### Stability of L-Cysteine Hydrochloride Monohydrate Produces by a Process of the Invention

### Method

A 10-mL drug product containing 50 mg/mL L-cysteine hydrochloride monohydrate and water for injection (“WFI”), produced by the process of Example 1, was stored at various accelerated storage conditions for up to 9 months. Samples were taken at 1, 2, 3, 6 and 9 months and assayed for bacterial endotoxins, pH, heavy metals including aluminum, drug product assay, fill volume, particulate matter, sterility and impurities.

TABLE 1
Inverted 10-mL L-cysteine hydrochloride monohydrate
at 40° C. ± 2° C. 75% RH ± 5%

| | | Result | | | |
|---|---|---|---|---|---|
| Test | Acceptance Criteria | 1 Month | 2 Months | 3 Months | 6 Months |
| pH | 1.0 to 2.5 | 1.3 | 1.2 | 1.2 | 1.2 |
| Assay | 85% to 115% | 109.3% | 107.4% | 109.8% | 110.6% |
| | label claim | | | | |
| Total | Sum All | 0.7% | 0.8% | 0.8% | 0.9% |
| Impurity | Reported ≥0.05% | | | | |

TABLE 2
Inverted 50-mL L-cysteine hydrochloride monohydrate
at 40° C. ± 2° C. 75% RH ± 5%

| | | Result | | | |
|---|---|---|---|---|---|
| | | 1 | 2 | 3 | 6 |
| Test | Acceptance Criteria | Month | Months | Months | Months |
| pH | 1.0 to 2.5 | 1.3 | 1.2 | 1.3 | 1.2 |
| Assay | 85% to 115% label claim | 108.5% | 107.4% | 107.9% | 108.4% |
| Total | Sum All Reported ≥0.05% | 1.2% | 1.1% | 1.2% | 1.4% |
| Impurity | | | | | |

TABLE 3
Upright 10-mL L-cysteine hydrochloride monohydrate
at 25° C. ± 2° C. 60% RH ± 5%

| | | Result | | |
|---|---|---|---|---|
| Test | Acceptance Criteria | 3 Month | 6 Month | 9 Month |
| pH | 1.0 to 2.5 | 1.2 | 1.2 | 1.2 |
| Assay | 85% to 115% label claim | 110.2% | 110.0% | 109.6% |
| Total Impurity | Sum All Reported ≥0.05% | 0.7% | 0.9% | 0.8% |

TABLE 4
Invert 10-mL L-cysteine hydrochloride monohydrate
at 25° C. ± 2° C. 60% RH ± 5%

| | | Result | | |
|---|---|---|---|---|
| Test | Acceptance Criteria | 3 Month | 6 Month | 9 Month |
| pH | 1.0 to 2.5 | 1.2 | 1.2 | 1.2 |
| Assay | 85% to 115% label claim | 110.9% | 110.1% | 110.2% |
| Total Impurity | Sum All Reported ≥0.05% | 0.6% | 0.7% | 0.7% |

TABLE 5
Inverted 10-mL L-cysteine hydrochloride monohydrate
at 30° C. ± 2° C. 60% RH ± 5%

| | | Result | | |
|---|---|---|---|---|
| Test | Acceptance Criteria | 3 Month | 6 Month | 9 Month |
| pH | 1.0 to 2.5 | 1.2 | 1.2 | 1.2 |
| Assay | 85% to 115% label claim | 108.9% | 110.7% | 109.0% |
| Total Impurity | Sum All Reported ≥0.05% | 0.8% | 0.8% | 0.8% |

### Results

As shown in Tables 1-5, the 10-mL and 50-mL drug product containing 50 mg/mL L-cysteine hydrochloride monohydrate and WFI was stable at all storage condition and durations. Thus, drug product produced by the process of the present invention are free of the sterilization issues that accompany heat sensitive active pharmaceutical ingredients.

What is claimed is:

1. A process of manufacturing a pharmaceutical composition, the process comprising the following steps:
(i) adding a pharmaceutically acceptable vehicle to a batch container comprising:
(a) a top side comprising an opening; and
(b) a pressurizing means,
(ii) purging the batch container with nitrogen gas;
(iii) mixing the drug vehicle;
(iv) adding an active pharmaceutical ingredient (API) to the pharmaceutically acceptable vehicle to create the pharmaceutical composition;
(v) mixing the pharmaceutical composition until the API is dissolved in the pharmaceutically acceptable vehicle;
(vi) sealing the opening of the top side of the batch container; and
(vii) pressurizing the batch container with nitrogen gas.

2. The process of claim 1, further comprising the following steps between (v) and (vi):
removing a portion of the pharmaceutical composition through a pharmaceutical composition removal means on the bottom side of the batch container;
adding the removed pharmaceutical composition to the opening of the top side of the batch container; and
adding additional pharmaceutically acceptable vehicle to the pharmaceutical composition to obtain a desired API concentration.

3. The process of claim 1, further comprising the following steps:
determining a dissolved oxygen content of the pharmaceutically acceptable vehicle after step (iii) and before step (iv); and
removing an amount of pharmaceutical composition necessary for assay and pH testing after step (viii) and before step (ix).

4. The process of claim 1, further comprising the following steps:
(xi) filling a product container with the pharmaceutical composition from the batch container using filling equipment;
(xii) stoppering the product container; and
(xiii) capping the product container.

5. The process of claim 1, further comprising the following steps each of which occurs prior to step (xi):
sterilizing stoppers in sterilization bags at 121° C. for 30 minutes;
wrapping filling equipment and sterilizing filling equipment at 121° C. for 30 minutes; and
washing vials with water for injection and depyrogenating vials in a depyrogenation oven.

6. The process of claim 1, wherein the batch container was pressurized to a p.s.i of about 5.

7. The process of claim 1, wherein the API is L-cysteine hydrochloride monohydrate.

8. The process of claim 7, wherein the API is at a concentration of about 50 milligrams per milliliter.

9. The process of claim 1, wherein the pharmaceutically acceptable vehicle is water for injection.

10. A sterile pharmaceutical composition prepared by the process of claim 1.

11. The composition of claim 10, wherein the API is L-cysteine hydrochloride monohydrate.

12. The composition of claim 11, wherein the desired API concentration is 50 milligrams per milliliter.

13. The composition of claim 11, wherein the pharmaceutically acceptable vehicle is water for injection.

14. The composition of claim 13, wherein the composition has a pH form about 1.0 to about 2.5.

15. The composition of claim 14, wherein the composition comprises less than about 1.2% cystine.

16. A container comprising a composition of claim 10.

17. The container of claim 16, wherein the container is a vial.

18. A sterile pharmaceutical composition prepared by a process comprising the following steps:
(i) adding a pharmaceutically acceptable vehicle to a batch container comprising:
(a) a bottom side comprising a pharmaceutical composition removal means;
(b) a top side comprising an opening; and
(c) a pressurizing means,
(ii) purging the batch container with nitrogen gas;
(iii) mixing the drug vehicle;
(iv) adding an active pharmaceutical ingredient (API) to the pharmaceutically acceptable vehicle to create the pharmaceutical composition;
(v) mixing the pharmaceutical composition until the API is dissolved in the pharmaceutically acceptable vehicle;
(vi) sealing the opening of the top side of the batch container;
(vii) pressurizing the batch container with nitrogen gas;
(viii) filling a product container with the pharmaceutical composition from the batch container using filling equipment;
(ix) stoppering the product container; and
(x) capping the product container,
wherein prior to step (viii) the following steps occur:
sterilizing stoppers in sterilization bags at 121° C. for 30 minutes;
wrapping filling equipment and sterilizing filling equipment at 121° C. for 30 minutes; and
washing vials with water for injection and depyrogenating vials in a depyrogenation oven.

19. A container comprising a sterile pharmaceutical composition prepared by a process comprising the following steps:
(i) adding a pharmaceutically acceptable vehicle to a batch container comprising:
(a) a bottom side comprising a pharmaceutical composition removal means;
(b) a top side comprising an opening; and
(c) a pressurizing means, (ii) purging the batch container with nitrogen gas;
(iii) mixing the drug vehicle;
(iv) adding an active pharmaceutical ingredient (API) to the pharmaceutically acceptable vehicle to create the pharmaceutical composition;
(v) mixing the pharmaceutical composition until the API is dissolved in the pharmaceutically acceptable vehicle;
(vi) sealing the opening of the top side of the batch container;
(vii) pressurizing the batch container with nitrogen gas;
(viii) filling a product container with the pharmaceutical composition from the batch container using filling equipment;
(ix) stoppering the product container; and
(x) capping the product container,
wherein prior to step (viii) the following steps occur:
sterilizing stoppers in sterilization bags at 121° C. for 30 minutes;
wrapping filling equipment and sterilizing filling equipment at 121° C. for 30 minutes; and
washing vials with water for injection and depyrogenating vials in a depyrogenation oven.
